(19) United States
(12) Patent Application Publication
(10) Pub. No.: US 2020/0392231 A1
(43) Pub. Date: Dec. 17, 2020

(54) Methods of Managing Hepatic Steatosis

(71) Applicant: Emory University, Atlanta, GA (US)

(72) Inventors: Frank Anania, Atlanta, GA (US); Reben Raeman, Atlanta, GA (US)

(21) Appl. No.: 16/770,217

(22) PCT Filed: Dec. 6, 2018
(86) PCT No.: PCT/US2018/064191

§ 371 Date: Jun. 5, 2020

Related U.S. Application Data
(60) Provisional application No. 62/595,571, filed on Dec. 6, 2017.

(51) Int. Cl. C07K 16/28; A61P 1/16

(52) U.S. Cl. CPC C07K 16/2839; A61P 1/16; A61K 2039/505; C07K 2317/76; C07K 2317/24

(57) ABSTRACT

This disclosure relates to methods of using a specific binding agent that targets alpha4 beta7 integrin to treat or prevent fatty liver diseases such as, hepatic steatosis, nonalcoholic fatty liver disease (NAFLD) and nonalcoholic steatohepatitis (NASH). In certain embodiments, a specific binding agent that targets alpha4 beta7 integrin is an antibody. In certain embodiments, antibody that binds alpha4 beta7 integrin is vedolizumab. In certain embodiments, the subject is or is not diagnosed with inflammatory bowel disease.

### CROSS-REFERENCE TO RELATED APPLICATIONS

This application claims the benefit of U.S. Provisional Application No. 62/595,571 filed Dec. 6, 2017. The entirety of this application is hereby incorporated by reference for all purposes.

### STATEMENT REGARDING FEDERALLY FUNDED RESEARCH

This invention was made with government support under DK110264, DK062092, and I01BX001746 awarded by the NIH and VA. The government has certain rights in the invention.

### BACKGROUND

Fatty liver, or hepatic steatosis, is a term that describes the buildup of fat in the liver. Excessive alcohol use causes fat to accumulate, damages the liver, and cirrhosis may develop. Nonalcoholic fatty liver disease (NAFLD) is a fatty liver disease associated with obesity related disorders, such as type-2 diabetes and metabolic syndrome, occurring in people who drink little or no alcohol. At the beginning of the NAFLD spectrum is simple steatosis, which is characterized by a build-up of fat within the liver. Liver steatosis without inflammation is usually benign and slow or non-progressive. Nonalcoholic steatohepatitis (NASH) is a more advanced and severe subtype of NAFLD where steatosis is complicated by liver-cell injury and inflammation, with or without fibrosis. NASH results in liver damage characteristic of alcoholic liver disease; however, it occurs in people who drink little or no alcohol. NASH can be severe and can lead to cirrhosis, in which the liver is permanently damaged and scarred and no longer able to work properly. Liver biopsy is typical to make a definitive diagnosis of NASH.

Chronic liver inflammation is a precursor to fibrosis, which can progress to cirrhosis, end-stage liver disease and hepatocellular carcinoma. In addition to insulin resistance, altered lipid storage and metabolism, accumulation of cholesterol within the liver, oxidative stress resulting in increased hepatic injury, and bacterial translocation secondary to disruption of gut microbiota (associated with high fructose-containing diet) have all been implicated as important co-factors contributing to progression of NASH. Due to the growing epidemic of obesity and diabetes, NASH is projected to become the most common cause of advanced liver disease and the most common indication for liver transplantation. The burden of NASH, combined with a lack of any approved therapeutic interventions, represents an unmet medical need.

Lefebvre report using cenicriviroc for the treatment of fibrosis, NASH, and NAFLD. See U.S. Patent Application Publication Nos. 2017/0239262 and 2017/0319548. Bisgaier et al. report the treatment of NASH with gemcabene. See U.S. Patent Application Publication No. 2017/0172954.

Chao et al. report the co-existence of non-alcoholic fatty liver disease and inflammatory bowel disease. World J Gastroenterol, 2016, 22(34): 7727-7734. Ali et al. report on research on the treatment of primary sclerosing cholangitis. Intractable & Rare Diseases Research. 2015, 4(1):1-6.

Vedolizumab is a humanized monoclonal antibody that is an integrin receptor antagonist and inhibits the migration of memory T-lymphocytes across the endothelium into inflamed gastrointestinal parenchymal tissue. ENTYVIO (vedolizumab) is indicated for the treatment of ulcerative colitis and Crohn's disease. See also ENTYVIO (vedolizumab), FDA product label, 2014; U.S. Pat. Nos. 7,147,851, 7,368,543; and U.S. Patent Publication No. US2012/0282249.

Referenced cited herein are not an admission of prior art.

### SUMMARY

This disclosure relates to methods of using a specific binding agent that targets alpha4 beta7 integrin to treat or prevent fatty liver diseases such as, hepatic steatosis, nonalcoholic fatty liver disease (NAFLD) and nonalcoholic steatohepatitis (NASH). In certain embodiments, a specific binding agent that targets alpha4 beta7 integrin is an antibody. In certain embodiments, antibody that binds alpha4 beta7 integrin is vedolizumab. In certain embodiments, the subject is or is not diagnosed with inflammatory bowel disease.

In certain embodiments, the disclosure relates to methods of treating hepatic steatosis or NAFLD comprising administering an effective amount of a specific binding agent that targets alpha4 beta7 integrin to a subject in need thereof. In certain embodiments, the subject is at risk of, exhibiting symptoms, or diagnosed with NAFLD or metabolic syndrome.

In certain embodiments, the disclosure relates to methods of treating or preventing NASH by administering an effective amount a specific binding agent that targets alpha4 beta7 integrin to a subject in need thereof. In certain embodiments, the subject is at risk of, exhibiting symptoms, or diagnosed with NASH. In certain embodiments, the disclosure relates to methods of preventing NASH by administering an effective amount a specific binding agent that targets alpha4 beta7 integrin to a subject in need thereof. In certain embodiments, the subject is diagnosed with NAFLD.

In certain embodiments, the disclosure relates to methods of treating alcohol induced fatty liver disease comprising administering an effective amount of a specific binding agent that targets alpha4 beta7 integrin to a subject in need thereof.

### BRIEF DESCRIPTION OF THE DRAWINGS

FIG. 1A illustrates an experiment. F11r^{-/-} mice were fed a HFCD diet and administered an anti-alpha4 beta7 antibody or IgG as a control.

FIG. 1B shows data on total CD4 positive T cells in prayer's patch after administration.

FIG. 1C shows data on total CD4 positive and alpha4 beta7 positive cells in prayer's patch after administration.

FIG. 1D shows data on the liver enzyme alanine aminotransferase (ALT) after administration.

FIG. 1E shows data on the liver enzyme alanine aminotransferase aspartate aminotransferase (AST) after administration.

FIG. 1F shows data on liver weight as a percentage of body weight after administration.

FIG. 1G shows data on cholesterol levels after administration.

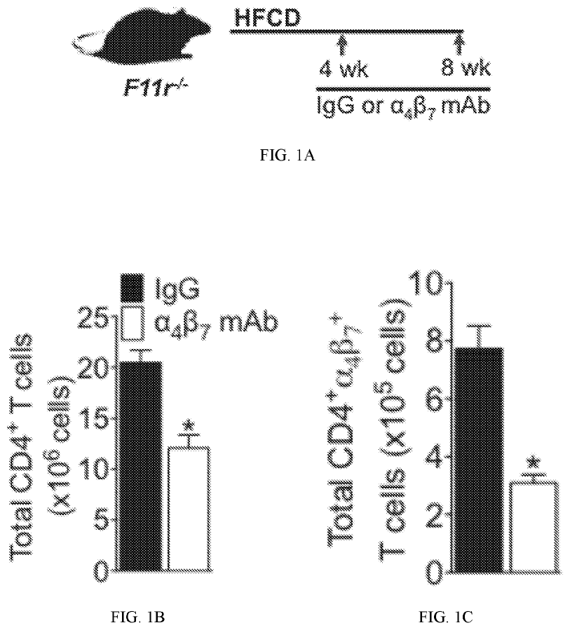

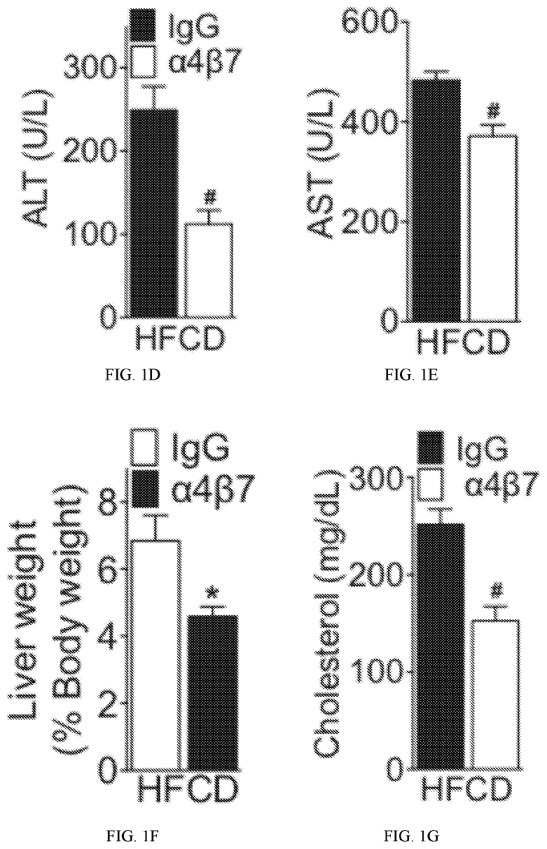

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

“Treating” includes ameliorating, mitigating, and reducing the instances of a disease or condition, or the symptoms of a disease or condition.

“Administering” includes any mode of administration, such as oral, subcutaneous, sublingual, transmucosal, parenteral, intravenous, intra-arterial, buccal, sublingual, topical, vaginal, rectal, ophthalmic, nasal, inhaled, and transdermal. “Administering” can also include prescribing or filling a prescription for a dosage form comprising a particular compound. “Administering” can also include providing directions to carry out a method involving a particular compound or a dosage form comprising the compound.

“Therapeutically effective amount” means the amount of an active substance that, when administered to a subject for treating a disease, disorder, or other undesirable medical condition, is sufficient to have a beneficial effect with respect to that disease, disorder, or condition. The therapeutically effective amount will vary depending on the chemical identity and formulation form of the active substance, the disease or condition and its severity, and the age, weight, and other relevant characteristics of the patient to be treated.

### Specific Binding Agents and Antibodies to Alpha4 Beta7

With regard to any of the methods disclosed herein, the specific binding agent that targets alpha4 beta7 may be an antibody that binds integrin alpha4 beta7. In certain embodiments, that antibody that binds integrin alpha4 beta7 is vedolizumab. Method of making and using vedolizumab and other anti-alpha4 beta7 antibodies are provide for in U.S. Patent Application Pub. No.: US2012/0282249.

The cell surface molecule, “alpha4 beta7 integrin,” or “alpha4 beta7,” is a heterodimer of an alpha 4 chain (CD49D, ITGA4) and a beta 7 chain (ITGB7). Each chain can form a heterodimer with an alternative integrin chain, to form alph4beta1 or alphEbeta7. Human alpha 4 and beta 7 genes (GenBank (National Center for Biotechnology Information, Bethesda, Md.) RefSeq Accession numbers NM-000885 and NM-000889, respectively) are expressed by B and T lymphocytes, particularly memory CD4+ lymphocytes. Typical of many integrins, alpha4 beta7 can exist in either a resting or activated state. Ligands for alpha4 beta7 include vascular cell adhesion molecule (VCAM), fibronectin and mucosal addressin (MAdCAM (e.g., MAdCAM-1)).

As used herein, a specific binding agent for the alpha4 beta7 complex” binds stronger to alpha4 beta7, compared to alpha4 beta1 or alphaE beta7. Suitable specific binding agents may be prepared using methods known in the art. An exemplary alpha4 beta7 polypeptide specific binding agent of the present disclosure is capable of binding a certain portion of the alpha4 beta7 polypeptides, and preferably modulating the activity or function of alpha4 beta7 polypeptides. Specific binding agents such as antibodies and antibody fragments that specifically bind alpha4 beta7 polypeptides are within the scope of the present disclosure. The antibodies may be polyclonal including mono-specific polyclonal, monoclonal (mAbs), recombinant, chimeric, humanized such as CDR-grafted, human, single chain, catalytic, multi-specific and/or bi-specific, as well as antigen-binding fragments, variants, and/or derivatives thereof.

Once a polynucleotide sequences are identified which encode each chain of the full length monoclonal antibody or the Fab or Fv fragment(s) of the disclosure, host cells, either eukaryotic or prokaryotic, may be used to express the monoclonal antibody polynucleotides using recombinant techniques well known and routinely practiced in the art. Alternatively, transgenic animals are produced wherein a polynucleotide encoding the desired specific binding agent is introduced into the genome of a recipient animal, such as, for example, a mouse, rabbit, goat, or cow, in a manner that permits expression of the polynucleotide molecules encoding a monoclonal antibody or other specific binding agent. In one aspect, the polynucleotides encoding the monoclonal antibody or other specific binding agent can be ligated to mammary-specific regulatory sequences, and the chimeric polynucleotides can be introduced into the germline of the target animal. The resulting transgenic animal then produces the desired antibody in its milk [Pollock et al., J Immunol Meth 231:147-157 (1999); Little et al., Immunol Today 8:364-370 (2000)]. In addition, plants may be used to express and produce JAML or JAML D1 specific binding agents such as monoclonal antibodies by transfecting suitable plants with the polynucleotides encoding the monoclonal antibodies or other specific binding agents.

In another embodiment of the present disclosure, a monoclonal or polyclonal antibody or fragment thereof that is derived from other than a human species may be “humanized” or “chimerized”. Methods for humanizing non-human antibodies are well known in the art. (see U.S. Pat. Nos. 5,859,205, 5,585,089, and 5,693,762). Humanization is performed, for example, using methods described in the art [Jones et al., Nature, 321: 522-525 (1986); Riechmann et al., Nature, 332: 323-327 (1988); Verhoeyen et al., Science, 239:1534-1536 (1988)] by substituting at least a portion of, for example a rodent, complementarity-determining region (CDRs) for the corresponding regions of a human antibody. The disclosure also provides variants and derivatives of these human antibodies as discussed herein and well known in the art.

Also encompassed by the disclosure are fully human antibodies that bind alpha4 beta7 polypeptides, as well as, antigen-binding fragments, variants and/or derivatives thereof. Alternatively, transgenic animals (e.g., mice) that are capable of producing a repertoire of human antibodies in the absence of endogenous immunoglobulin production can be used to generate such antibodies. This can be accomplished by immunization of the animal with an alpha4 beta7 antigen or fragments thereof where the alpha4 beta7 fragments have an amino acid sequence that is unique to alpha4 beta7. Such immunogens can be optionally conjugated to a carrier. See, for example, Jakobovits et al., Proc Natl Acad Sci (USA), 90: 2551-2555 (1993); Jakobovits et al., Nature 362: 255-258 (1993); Bruggermann et al., Year in Immuno, 7: 33 (1993). In one method, such transgenic animals are produced by incapacitating the endogenous loci encoding the heavy and light immunoglobulin chains therein, and inserting loci encoding human heavy and light chain proteins into the genome thereof. Partially modified animals, that are those having less than the full complement of these modifications, are then crossbred to obtain an animal having all of the desired immune system modifications. When administered an immunogen, these transgenic animals are capable of producing antibodies with human variable regions, including human (rather than e.g., murine) amino acid sequences, that are immuno-specific for the desired antigens. See PCT application Nos., PCT/US96/05928 and PCT/US93/06926. Additional methods are described in U.S. Pat. No. 5,545,807, PCT application Nos. PCT/US91/245, PCT/GB89/01207, and in EP 546073B1 and EP 546073A1. Human antibodies may also be produced by the expression of recombinant DNA in host cells or by expression in hybridoma cells as described herein.

Transgenesis is achieved in a number of different ways. See, for example, Bruggeman et al., Immunol Today 17:391-7 (1996). In one approach, a minilocus is constructed such that gene segments in a germline configuration are brought artificially close to each other. Due to size limitations (i.e., having generally less than 30 kb), the resulting minilocus will contain a limited number of differing gene segments, but is still capable of producing a large repertoire of antibodies. Miniloci containing only human DNA sequences, including promoters and enhancers are fully functional in the transgenic mouse.

Once an appropriate transgenic mouse (or other appropriate animal) has been identified, using any of the techniques known in the art to detect serum levels of a circulating antibody (e.g., ELISA), the transgenic animal is crossed with a mouse in which the endogenous Ig locus has been disrupted. The result provides progeny wherein essentially all B cells express human antibodies.

As still another alternative, the entire animal Ig locus is replaced with the human Ig locus, wherein the resulting animal expresses only human antibodies. In another approach, portions of the animal's locus are replaced with specific and corresponding regions in the human locus. In certain cases, the animals resulting from this procedure may express chimeric antibodies, as opposed to fully human antibodies, depending on the nature of the replacement in the mouse Ig locus.

Human antibodies can also be produced by exposing human splenocytes (B or T cells) to an antigen in vitro, then reconstituting the exposed cells in an immunocompromised mouse, e.g. SCID or nod/SCID. See Brams et al., J Immunol, 160: 2051-2058 [1998]; Carballido et al., Nat Med, 6: 103-106 [2000]. In one approach, engraftment of human fetal tissue into SCID mice (SCID-hu) results in long-term hematopoiesis and human T-cell development [McCune et al., Science 241:1532-1639 (1988); Ifversen et al., Sem Immunol 8:243-248 (1996)]. Any humoral immune response in these chimeric mice is completely dependent on co-development of T-cells in the animals [Martensson et al., Immunol 83:1271-179 (1994)]. In an alternative approach, human peripheral blood lymphocytes are transplanted intraperitoneally (or otherwise) into SCID mice [Mosier et al., Nature 335:256-259 (1988)]. When the transplanted cells are treated with either a priming agent, such as Staphylococcal Enterotoxin A (SEA) [Martensson et al., Immunol 84: 224-230 (1995)], or anti-human CD40 monoclonal antibodies [Murphy et al., Blood 86:1946-1953 (1995)], higher levels of B cell production are detected.

Alternatively, an entirely synthetic human heavy chain repertoire is created from unrearranged V gene segments by assembling each human VH segment with D segments of random nucleotides together with a human J segment [Hoogenboom et al., J Mol Biol 227:381-388 (1992)]. Likewise, a light chain repertoire is constructed by combining each human V segment with a J segment [Griffiths et al., EMBO J. 13:3245-3260 (1994)]. Nucleotides encoding the complete antibody (i.e., both heavy and light chains) are linked as a single chain Fv fragment and this polynucleotide is ligated to a nucleotide encoding a filamentous phage minor coat protein. When this fusion protein is expressed on the surface of the phage, a polynucleotide encoding a specific antibody is identified by selection using an immobilized antigen.

In still another approach, antibody fragments are assembled as two Fab fragments by fusion of one chain to a phage protein and secretion of the other into bacterial periplasm [Hoogenboom et al., Nucl Acids Res 19:4133-4137 [1991]; Barbas et al., Proc Natl Acad Sci (USA) 88:7978-7982 (1991)].

Large-scale production of chimeric, humanized, CDR-grafted, and fully human antibodies, or antigen-binding fragments thereof, are typically produced by recombinant methods.

Polynucleotide molecule(s) encoding the heavy and light chains of each antibody or antigen-binding fragments thereof, can be introduced into host cells and expressed using materials and procedures described herein. In a preferred embodiment, the antibodies are produced in mammalian host cells, such as CHO cells.

The specific binding agents of the present disclosure, such as the antibodies, antibody fragments, and antibody derivatives of the disclosure can further comprise any constant region known in the art. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. In one embodiment, the light or heavy chain constant region is a fragment, derivative, variant, or mutant of a naturally occurring constant region.

In one embodiment, the specific binding agents of the present disclosure, such as the antibodies, antibody fragments, and antibody derivatives of the disclosure comprise an IgG.

Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype. See also Lantto et al., 2002, Methods Mol. Biol. 178:303-16.

The specific binding agents of the present disclosure, such as the antibodies, antibody fragments, and antibody derivatives of the disclosure may comprise the IgG1 heavy chain constant domain or a fragment of the IgG1 heavy chain domain. The antibodies, antibody fragments, and antibody derivatives of the disclosure may further comprise the constant light chain kappa or lambda domains or a fragment of these. Light chain constant regions and polynucleotides encoding them are provided herein below. In another embodiment, the antibodies, antibody fragments, and antibody derivatives of the disclosure further comprise a heavy chain constant domain, or a fragment thereof, such as the IgG2 heavy chain constant region.

### Alpha4 Beta7 Blockade Reverses Microbial Dysbiosis and Attenuates NASH and Metabolic Syndrome

A western diet (high-fat, high-fructose, and high-cholesterol diet, HFCD)-induces a decrease in intestinal epithelial barrier integrity and plays a role in the heightened inflammatory response that underlies NASH. Specifically, HFCD diet increased gut permeability results in severe NASH in mice with a compromised intestinal epithelial barrier (F11r−/− knockout mice). These pathological changes were triggered by gut dysbiosis and mucosal inflammation, facilitating translocation of gut bacterial endotoxin, a potent inducer of hepatic inflammation. Humans with nonalcoholic fatty liver disease (NAFLD) but with no known inflammatory bowel disease (IBD) demonstrated increased colonic inflammation and decreased F11r expression in the colonic mucosa. Together, these data indicate a role of HFCD-induced intestinal mucosal inflammation in NASH pathogenesis, but the underlying mechanistic details are not well understood.

Experiments described herein were performed to elucidate the role of CD4 T cells in intensifying intestinal epithelial barrier disruption in NASH. Data indicates a significant microbiota driven increase in the infiltration and accumulation of inflammatory CD4 T cells in the colonic mucosa of F11r−/− mice fed a WD. The infiltration of inflammatory CD4 T cells in the colonic mucosa is mediated through gut homing integrin receptor alpha4 beta7 and its ligand MAdCAM-1, as blocking alpha4 beta7 significantly reduced CD4 T cell infiltration in the colonic mucosa, attenuated mucosal inflammation and improved intestinal epithelial barrier. Alpha4 beta7 blockade also reversed microbial dysbiosis and attenuated NASH and metabolic syndrome. These findings indicate a role of alpha4 beta7-mediated infiltration of inflammatory CD4 T cells in mucosal inflammation in NASH and provide a therapy based upon alpha4 beta7 blockade.

Male C57BL/6j (control) or F11r−/− mice were fed either normal diet (ND) or the western diet (WD) for 8 weeks. Liver and mucosal injury and inflammation were assessed by histological, RT-qPCR, and flow cytometric analysis. F11r−/− mice fed the WD developed severe histological, biochemical, and metabolic characteristics of human NASH as well as microbial dysbiosis, mucosal inflammation, and colonic epithelial barrier disruption. Flow cytometry revealed a significant increase in the infiltration of inflammatory CD4 T cells (CD4+PD-1+CD44+) in the colonic mucosa of F11r−/− mice compared to controls. Infiltration of inflammatory CD4 T cells in the colonic mucosa of these mice correlated with microbiota dependent increase in the expression of mucosal addressin cell adhesion molecule (MAdCAM)-1, ligand for the gut-homing integrin receptor alpha4beta7. Strikingly, WD also induced a systemic increase in alpha4beta7+ inflammatory CD4 T cells in F11r−/− WD mice suggesting alpha4beta7/MAdCAM-1-mediated infiltration and accumulation of inflammatory CD4 T cells in the colonic mucosa.

Correspondingly, abolishing the alpha4beta7/MAdCAM-1 association with an alpha4 beta7 blocking antibody, significantly reduced accumulation of CD4 T cells in the colonic mucosa, improved colonic epithelial barrier function, and attenuated NASH and indices of the metabolic syndrome. This data demonstrate a role for alpha4 beta7-mediated infiltration and accumulation of CD4 T cells in mucosal inflammation in NASH pathogenesis and provide a framework for targeted therapy for alpha4 beta7 blockade to attenuate NASH progression.

### Methods of Use

This disclosure relates to methods of using a specific binding agent that targets alpha4 beta7 or an antibody that binds alpha4 beta7 integrin such as vedolizumab to treat or prevent fatty liver diseases such as hepatic steatosis, nonalcoholic fatty liver disease (NAFLD) and nonalcoholic steatohepatitis (NASH). With regard to any of the methods disclosed herein, the specific binding agent that targets alpha4 beta7 may be an antibody that binds alpha4 beta7 integrin. In certain embodiments, antibody that binds alpha4 beta7 integrin is vedolizumab.

In certain embodiments, the disclosure relates to methods of treating hepatic steatosis or NAFLD comprising administering an effective amount of a specific binding agent that targets alpha4 beta7 to a subject in need thereof. In certain embodiments, the subject is at risk of, exhibiting symptoms, or diagnosed with NAFLD or metabolic syndrome.

In certain embodiments, the disclosure relates to methods of treating NASH by administering an effective amount a specific binding agent that targets alpha4 beta7 to a subject in need thereof. In certain embodiments, the subject is at risk of, exhibiting symptoms, or diagnosed with NASH. In certain embodiments, the disclosure relates to methods of preventing NASH by administering an effective amount a specific binding agent that targets alpha4 beta7 to a subject in need thereof. In certain embodiments, the subject is diagnosed with NAFLD.

In certain embodiments, a subject is at risk of NAFLD due to obesity, insulin resistance, an enlarged liver, signs of cirrhosis, or abnormal levels of liver enzymes, triglycerides and/or cholesterol. Signs of insulin resistance include darkened skin patches over your knuckles, elbows, and knees. Signs of cirrhosis include jaundice, a condition that causes your skin and whites of your eyes to turn yellow. A sign of NAFLD or NASH includes blood test showing increased levels of the liver enzymes alanine aminotransferase (ALT) and aspartate aminotransferase (AST). An enlarged liver or an abnormal amount of fat in a liver may be identified by ultrasound, computerized tomography (CT) scans, magnetic resonance imaging or combinations thereof. A liver biopsy may be used to detect liver inflammation and damage to diagnose NASH.

Metabolic syndrome is typically diagnosed in the presence of three or more of the following medical issues: large waste size, e.g., 40 inches or more, high triglycerides e.g., triglyceride level of 150 mg/dL or higher, low levels of HDL cholesterol less than 50 mg/dL, high blood pressure, e.g., 130/85 mmHg or higher, and high blood glucose (or blood sugar) levels, a fasting blood sugar level of 100 mg/dL or higher.

In certain embodiments, this disclosure relates to methods of treating fibrosis or a fibrotic disease or condition in a subject in need thereof comprising administering to the subject a therapeutically effective amount of a pharmaceutical composition comprising a specific binding agent that targets alpha4 beta7 integrin or an antibody that binds alpha4 beta7 integrin such as vedolizumab. In certain embodiments, the fibrosis or fibrotic disease or condition is liver fibrosis or renal fibrosis. In certain embodiments, the liver fibrosis is associated with fatty liver diseases such as, hepatic steatosis, NASH or NAFLD.

Fibrosis is the formation of excess fibrous connective tissue in an organ or tissue in a reparative or reactive process. This can be a reactive, benign, or pathological state. The deposition of connective tissue in the organ and/or tissue can obliterate the architecture and function of the underlying organ or tissue. Fibrosis is this pathological state of excess deposition of fibrous tissue, as well as the process of connective tissue deposition in healing.

Fibrosis is similar to the process of scarring, in that both involve stimulated cells laying down connective tissue, including collagen and glycosaminoglycans. Hepatocyte damage resulting from factors such as fat accumulation, viral agents, excessive alcohol consumption, hepatotoxins, inevitably triggers an inflammatory immune response. The increased production of cytokines and chemokines in the liver leads to recruitment of pro-inflammatory monocytes (precursor cells) that subsequently mature into pro-inflammatory macrophages. Pro-inflammatory macrophages are pro-fibrogenic in nature and ultimately lead to the activation of hepatic stellate cells (HSCs) that are primarily responsible for the deposition of extracellular matrix (ECM).

Infiltration of various immune cell populations, resulting in inflammation, is a pathogenic feature following acute- and chronic liver injury. Chronic liver inflammation leads to continuous hepatocyte injury which can lead to fibrosis and cirrhosis. Interactions between intra-hepatic immune cells lead to increased activation and migration of Kupffer cells and HSCs. Disruption of the intestinal microbiota and its downstream effects on the gut-liver axis both play an important role in metabolic disorders such as obesity, NAFLD and NASH.

The activation of hepatic stellate cells (HSCs) plays an important role in the pathogenesis of hepatic fibrosis. Following liver injury, hepatic stellate cells (HSCs) become activated and express a combination of matrix metalloproteinases (MMPs) and their specific tissue inhibitors (TIMPs). In the early phases of liver injury. HSCs transiently express MMP-3, MMP-13, and uro-plasminogen activator (uPA) and exhibit a matrix-degrading phenotype. Activated HSCs can amplify the inflammatory response by inducing infiltration of mono- and polymorphonuclear leucocytes. Infiltrating monocytes and macrophages participate in the development of fibrosis via several mechanisms, including increased secretion of cytokines and generation of oxidative stress-related products. In human liver diseases, increased MCP-1 is associated with macrophage recruitment and severity of hepatic fibrosis and primary biliary cirrhosis.

In the later stages of liver injury and HSC activation, the pattern changes and the cells express a combination of MMPs that have the ability to degrade normal liver matrix, while inhibiting degradation of the fibrillary collagens that accumulate in liver fibrosis. This pattern is characterized by the combination of pro-MMP-2 and membrane type 1 (MT1)-MMP expression, which drive pericellular generation of active MMP-2 and local degradation of normal liver matrix. In addition, there is a marked increase in expression of TIMP-1 leading to a more global inhibition of degradation of fibrillar liver collagens by interstitial collagenases (MMP-1/MMP-13). In liver injury associated with chronic alcoholic liver disease, the production of TNF-α, IL-1, IL-6, as well as the chemokine IL-8/CXCL8 is increased. TNF-α is also an important mediator of non-alcoholic fatty liver disease. These pathways play a significant role in the progression of liver fibrosis.

Inhibiting the activation of HSCs and accelerating the clearance of activated HSCs may be effective strategies for resolution of hepatic fibrosis.

In some embodiments, the present disclosure provides for methods of treating subjects at risk of developing liver fibrosis or cirrhosis. In further embodiments, liver fibrosis is associated with emerging cirrhosis. In some embodiments, the fibrosis or cirrhosis is associated with alcohol damage. In further embodiments, the cirrhosis is associated with a hepatitis infection, including but not limited to hepatitis B and hepatitis C infections, primary biliary cirrhosis (PBC), primary sclerosing cholangitis, or fatty liver disease.

In another embodiment, the fibrosis comprises non-cirrhotic hepatic fibrosis. In another further embodiment, the subject is infected by human immunodeficiency virus (HIV). In yet a further embodiment, the subject is infected with a hepatitis virus, including but not limited to HCV (hepatitis C virus). In further embodiment, the subject has diabetes. In a further embodiment, the subject has type 2 diabetes. In a further embodiment, the subject has type 1 diabetes. In a further embodiment, the subject has metabolic syndrome (MS). In further embodiments, the subject has one or more of these diseases or disorders. In a further embodiment, the subject is at risk of developing one or more of these diseases. In a further embodiment, the subject has insulin resistance. In further embodiments, the subject has increased blood glucose concentrations, high blood pressure, elevated cholesterol levels, elevated triglyceride levels, or is obese. In a further embodiment, the subject has polycystic ovary syndrome.

This disclosure provides methods of treating fibrosis such as hepatic steatosis or NASH associated with type 2 diabetes mellitus (T2DM) and metabolic syndrome (MS); HIV and HCV co-infection, or HCV infection. In another embodiment, the fibrosis comprises non-cirrhotic hepatic fibrosis. In another further embodiment, the subject is infected by human immunodeficiency virus (HIV). In yet a further embodiment, the subject is infected with a hepatitis virus, including but not limited to HCV (hepatitis C virus). In further embodiment, the subject has diabetes. In a further embodiment, the subject has type 2 diabetes. In a further embodiment, the subject has type 1 diabetes. In a further embodiment, the subject has metabolic syndrome (MS). In further embodiments, the subject has one or more of these diseases or disorders. In a further embodiment, the subject is at risk of developing one or more of these diseases. In a further embodiment, the subject has insulin resistance. In further embodiments, the subject has increased blood glucose concentrations, high blood pressure, elevated cholesterol levels, elevated triglyceride levels, or is obese. In a further embodiment, the subject has polycystic ovary syndrome.

In certain embodiments, this disclosure relates to methods of treating fibrosis or a fibrotic disease or condition in a subject in need thereof comprising administering to the subject a therapeutically effective amount of a pharmaceutical composition comprising a specific binding agent that targets alpha4 beta7 or an antibody that binds alpha4 beta7 integrin such as vedolizumab. In certain embodiments, the fibrosis or fibrotic disease or condition is liver fibrosis or renal fibrosis. In certain embodiments, the liver fibrosis is associated hepatic steatosis, NASH or NAFLD. In certain embodiments, the liver fibrosis is associated with emerging cirrhosis. In certain embodiments, the liver fibrosis comprises non-cirrhotic hepatic fibrosis. In certain embodiments, the subject is infected by human immunodeficiency virus (HIV). In certain embodiments, the subject has a disease or condition selected from the group consisting of alcoholic liver disease, HIV and HCV co-infection, viral hepatitis (such as HBV or HCV infection), type 2 diabetes mellitus (T2DM), metabolic syndrome (MS), and a combination thereof.

In certain embodiments, the specific binding agent that targets alpha4 beta7 integrin or an antibody that binds alpha4 beta7 integrin such as vedolizumab is co-administered with one or more additional active agents. In certain embodiments, the one or more additional active agents are one or more antiretroviral agents selected from the group consisting of entry inhibitors, nucleoside reverse transcriptase inhibitors, nucleotide reverse transcriptase inhibitors, non-nucleoside reverse transcriptase inhibitors, protease inhibitors, integrase inhibitors, maturation inhibitors, and combinations thereof.

In certain embodiments, the one or more additional antiretroviral agents are selected from the group consisting of lamivudine, efavirenz, raltegravir, bevirimat, alpha interferon, zidovudine, abacavir, lopinavir, ritonavir, tenofovir, tenofovir disoproxil, tenofovir prodrugs, emtricitabine, elvitegravir, cobicistat, darunavir, atazanavir, rilpivirine, dolutegravir, and a combination thereof. In certain embodiments, the one or more additional active agents are one or more immune system suppressing agents. In certain embodiments, the one or more additional active agents are selected from the group consisting of cyclosporine, tacrolimus, prednisolone, hydrocortisone, sirolimus, everolimus, azathioprine, mycophenolic acid, methotrexate, basiliximab, daclizumab, rituximab, anti-thymocyte globulin, anti-lymphocyte globulin, and a combination thereof.

In certain embodiments, methods disclosed herein comprise detecting a level of one or more biological molecules in the subject treated for fibrosis or the fibrotic disease or condition or condition, and determining a treatment regimen based on an increase or decrease in the level of one or more biological molecules, wherein the biological molecule is selected from the group consisting of lipopolysaccharide (LPS), LPs-binding protein (LBP), 16S rDNA, sCD14, intestinal fatty acid binding protein (I-FABP), zonulin-1, Collagen 1a1 and 3a1, TGF-beta, fibronectin-1, hs-CRP, IL-1beta, IL-6, IL-33, fibrinogen, MCP-1, MIP-1 alpha and -1beta, RANTES, sCD163, TGF-beta, TNF-alpha, a biomarker of hepatocyte apoptosis such as CK-18 (caspase-cleaved and total), and a combination thereof.

In certain embodiments, methods disclosed herein comprise detecting a level of one or biological molecules in the subject treated for fibrosis or the fibrotic disease or condition or condition, wherein an increase or decrease in the level of one or more biological molecules compared to a predetermined standard level is predictive of the treatment efficacy of fibrosis or the fibrotic disease or condition, wherein the biological molecule is selected from the group consisting of lipopolysaccharide (LPS), LPs-binding protein (LBP), 16S rDNA, sCD14, intestinal fatty acid binding protein (I-FABP), zonulin-1, Collagen 1a1 and 3a1, TGF-beta, fibronectin-1, hs-CRP, IL-1beta, IL-6, IL-33, fibrinogen, MCP-1, MIP-lalpha and -1beta, RANTES, sCD163, TGF-beta, TNF-alpha, a biomarker of hepatocyte apoptosis such as CK-18 (caspase-cleaved and total), and a combination thereof.

In certain embodiments, the one or more biological molecules are measured in a biological sample from a subject treated for fibrosis or the fibrotic disease or condition.

In certain embodiments, the biological sample is selected from blood, skin, hair follicles, saliva, oral mucous, vaginal mucous, sweat, tears, epithelial tissues, urine, semen, seminal fluid, seminal plasma, prostatic fluid, pre-ejaculatory fluid (Cowper's fluid), excreta, biopsy, ascites, cerebrospinal fluid, lymph, brain, and tissue extract sample or biopsy sample.

In certain embodiments, this disclosure relates to methods of delaying or preventing NASH comprising administering to a patient at risk of developing NASH a therapeutically effective amount of a pharmaceutical composition comprising vedolizumab, wherein delay or prevention of NASH is measured by changes from baseline in inflammatory biomarkers.

In certain embodiments, the level of inflammatory biomarker is increased or decreased after administration of the pharmaceutical composition compared with level of inflammatory biomarker at baseline.

In certain embodiments, this disclosure relates to methods of delaying or preventing NASH comprising administering to a patient at risk of developing NASH a therapeutically effective amount of a pharmaceutical composition comprising vedolizumab, wherein delay or prevention of NASH is measured by changes from baseline measurements of fibrosis.

In certain embodiments, the measurement of fibrosis is increased or decreased after administration of the pharmaceutical composition compared with measurement of fibrosis at baseline.

In one embodiment, the disclosure provides a method of treatment, wherein the vedolizumab is co-administered with one or more additional active agents. In a further embodiment, the one or more additional active agents are one or more antiretroviral agents selected from entry inhibitors, nucleoside reverse transcriptase inhibitors, nucleotide reverse transcriptase inhibitors, non-nucleoside reverse transcriptase inhibitors, protease inhibitors, integrase strand transfer inhibitors, maturation inhibitors, and combinations thereof. In a further embodiment, the one or more additional antiretroviral agents are selected from the group consisting of lamivudine, efavirenz, raltegravir, bevirimat, alpha interferon, zidovudine, abacavir, lopinavir, ritonavir, tenofovir, tenofovir disoproxil, tenofovir prodrugs, emtricitabine, elvitegravir, cobicistat, darunavir, atazanavir, rilpivirine, dolutegravir, and a combination thereof. In a further embodiment, the one or more additional active agents are one or more immune system suppressing agents. In a further embodiment, the one or more additional active agents are selected from the group consisting of cyclosporine, tacrolimus, prednisolone, hydrocortisone, sirolimus, everolimus, azathioprine, mycophenolic acid, methotrexate, basiliximab, daclizumab, rituximab, anti-thymocyte globulin, anti-lymphocyte globulin, and a combination thereof.

In certain embodiments, the disclosure provides methods for reducing the amount of liver fat or the accumulation of liver fat in the subject comprising administering vedolizumab alone or in combination with a statin. In some embodiments of the second aspect, the method is a method of treating a subject to reduce or prevent steatosis comprising administering vedolizumab alone or in combination with a statin. In some embodiments, the statin is atorvastatin, rosuvastatin, simvastatin, pravastatin, lovastatin, fluvastatin, or pitavastatin.

In certain embodiments, this disclosure relates to methods of treating fatty liver disease in the subject comprising administering vedolizumab alone or in combination with a statin. Another embodiment is a method of treating a liver disease where in the liver disease is hepatic steatosis or

NAFLD. In some embodiments, the method is a method of treating a subject to prevent or reduce the rate of progression of liver disease. In some embodiments, the liver disease is NASH. In some embodiments, the liver disease is alcoholic hepatic steatosis.

In any of the embodiments, the subject may have a risk factor for developing fatty liver (steatosis) wherein the risk factor is that the subject has metabolic syndrome, type-2 diabetes, impaired glucose tolerance, obesity, dyslipidemia, hepatitis B, hepatitis C, an HIV infection, or a metabolic disorder such as Wilson's disease, a glycogen storage disorder, or galactosemia. In some embodiments, the patient has diabetes mellitus. In some embodiments, the patient has an inflammatory condition. In some embodiments, the patient has an elevated body mass index above what is normal for gender, age and height.

### EXAMPLES

Colonic Mucosal Inflammation in HFCD-Fed F11r−/− Mice is Associated with a Significant Increase in the Quantity and Activation Status of CD4 T Cells.

Mice with a defect in intestinal epithelial barrier develop severe histological, biochemical, and metabolic characteristics of human NASH within eight weeks of feeding a HFCD. HFCD induced increase in intestinal epithelial permeability in these mice was associated with an increase in mucosal inflammation and intestinal epithelial barrier disruption. Since CD4 T cells play an important role in the maintenance of intestinal mucosal immune homeostasis, mucosal CD4 T cells were analyzed after 8 weeks of feeding the HFCD or the ND to F11r−/− or control mice. Both the percentage and the total number of CD4 T cells in the colonic mucosa of HFCD-fed F11r−/− mice were significantly higher than the control mice. A higher percentage of colonic CD4 T cells in HFCD fed F11r−/−mice expressed the T cell activation markers CD44 and programmed death (PD)-1 and the total number of inflammatory CD4 T cells (CD4+CD44+PD-1+) in the colonic mucosa was significantly higher in these mice. In contrast, HFCD diet only increased percentage, but not the total number of CD4 T cells in the control mice. The percentage and the total number of inflammatory CD4 T cells in the colonic mucosa of control mice fed the HFCD were also not significantly different from the ND fed mice. No differences in the CD4 T cell and inflammatory CD4 T cell populations were observed in the colonic mucosa of ND fed F11r−/− and control mice. The increased infiltration of inflammatory CD4 T cells in the colonic mucosa of F11r−/− mice correlated with intestinal epithelia barrier disruption as indicated by increased redistribution of tight junction proteins ZO-1 and occludin from the crypt area in the HFCD-fed F11r−/− mice. Together, these data demonstrate that HFCD induced increase in colonic CD4 T cell infiltration and activation may play a role in intestinal epithelial barrier disruption in NASH.

### Increase in Mucosa Associated Pro-Inflammatory Microbiota Promotes Mucosal MAdCAM-1 Expression in HFCD-Fed F11r−/− Mice

The integrin receptor alph4beta7 on CD4 T cells and its ligand MAdCAM-1 expressed on intestinal mucosa play a major role in CD4 T cell homing to colonic mucosa. Therefore, to determine whether HFCD-induced increased in CD4 T cell infiltration in the colonic mucosa was mediated through alph4beta7/MAdCAM-1, colonic mucosal tissue was stained for the expression of MAdCAM-1. HFCD increased MAdCAM-1 expression in the colonic mucosa of the control mice, however MAdCAM-1 expression was significantly higher in the F11r−/− mice fed the HFCD. Interestingly treatment with broad spectrum antibiotics, which attenuates mucosal inflammation and severity of NASH in HFCD-fed F11r−/− mice, reduced MAdCAM-1 expression in the colonic tissue of HFCD-fed F11r−/− mice suggesting a role of gut microbiota in regulating MAdCAM-1 expression in these mice. To further understand the microbiota associated with diet-induced increase in colonic MAdCAM-1 expression, colonic mucosa associated microbiota was analyzed using 16s rRNA sequencing followed by phylogenetic analysis, and a comparison of the microbial community structure using the unweighted UniFrac algorithm. Mucosa-associated microbiota were analyzed as they are considered to play a role in regulating mucosal homeostasis. Interestingly, similar to the microbiota observed in the luminal content of HFCD-fed F11r−/− mice, HFCD consumption resulted in marked decrease in Bacteroidetes and an increase in Proteobacteria in the F11r−/− mice relative to WT controls; a gut microbial composition distinctly linked to obesity and colitis. Additional analysis revealed a significant increase in desulfovibrionaceae, a bacterial taxa associated with colitis, and a decrease in Akkermansia, an anti-obesogenic, anti-inflammatory bacterial taxa, in the colonic mucosa of HFCD-fed F11r−/− mice. Together, these data indicate a direct link between pro-inflammatory mucosa-associated microbiota in HFCD-fed F11r−/− mice and excessive infiltration of CD4 T cells in the colonic mucosa.

### Diet Induced Infiltration of Inflammatory CD4 T Cells in the Colonic Mucosal Exacerbated Mucosal Inflammation in F11r−/− Mice

To ascertain the contribution of alph4beta7/MAdCAM-1 mediated colonic infiltration of inflammatory CD4 T cells in diet-induced intestinal epithelial barrier disruption in NASH, alph4beta7 integrin was blocked using a highly specific neutralizing monoclonal antibody (mAb) against alph4beta7 (Clone DATK32; Bioxcell, West Lebanon, N.H.). Alph4beta7 mAb treatment significantly reduced alph4beta7+CD4T cells in the Payer's patches and reduced CD4 T cell infiltration in the colonic lamina propria. Interestingly, alph4 beta7 mAb treatment did not affect colonic Treg population. Histological analysis of the colonic tissue revealed decreased immune cell infiltration and increased occludin and ZO-1 expression in the colonic mucosa of alph4 beta7 mAb treated mice suggesting that alph4beta7 mAb treatment was effective in reducing mucosal inflammation as well as restoring intestinal epithelial barrier in HFCD-fed F11r−/− mice.

### Blocking α4β7-Mediated Infiltration of CD4 T Cells in the Colonic Mucosa Reversed Steatohepatitis and Improved Metabolic Parameters in HFCD-Fed F11r−/− Mice

Histological analysis of liver tissue sections was performed to determine whether blocking alph4beta7 mediated colonic infiltration of inflammatory CD4 T cells also ameliorated hepatic steatosis, inflammation and fibrosis. Hepatic steatosis and inflammation assed by H&E staining as well as hepatic fibrosis assessed by Sirius Red staining was ameliorated by 4 weeks of a1ph4 beta7 mAb treatment in HFCD-fed F11r−/− mice. These results were further corroborated by a significant decrease in serum alanine aminotransferase (ALT) and aspartate aminotransferase (AST) levels, as well as a significant decrease in the expression of transcript levels of key molecules associated with hepatic fibrogenesis; alpha smooth muscle actin (aSMA), transforming growth factor β1 (TGFβ1), tissue inhibitor of metalloproteinase 1 (TIMP-1), and collagen I. Apart from attenuating steatohepatitis, four-weeks of a1ph4 beta7 mAb treatment also significantly improved metabolic parameters including body weight, liver and visceral fat weight expressed as percent of body weight, serum cholesterol levels and improved glucose tolerance and insulin sensitivity in HFCD-fed F11r−/− mice. Together, these data imply that ameliorating colonic mucosal inflammation and consequently improving intestinal epithelial barrier contributes to reversal of NASH.

1. A method of treating hepatic steatosis comprising administering an effective amount of a specific binding agent that targets alpha4 beta7 integrin to a subject in need thereof.

2. The method of claim 1, wherein the subject is at risk of, exhibiting symptoms, or diagnosed with NAFLD.

3. The method of claim 1, wherein the subject is at risk of, exhibiting symptoms, or diagnosed with or metabolic syndrome.

4. The method of claim 1, wherein the subject is diagnosed with alcohol induced fatty liver disease.

5. The method of claim 1, wherein the subject is diagnosed with insulin resistance.

6. The method of claim 1, wherein the subject is diagnosed with type 2 diabetes.

7. The method of claim 1, wherein the specific binding agent that targets alpha4 beta7 integrin is an antibody that binds alpha4 beta7 integrin.

8. The method of claim 7, wherein the antibody that binds alpha4 beta7 integrin is vedolizumab.

9. A method of treating or preventing nonalcoholic steatohepatitis (NASH) by administering an effective amount a specific binding agent that targets alpha4 beta7 integrin to a subject in need thereof.

10. The method of claim 9, wherein the subject is at risk of, exhibiting symptoms, or diagnosed with NASH or NAFLD.

11. The method of claim 9, wherein the specific binding agent that targets alpha4 beta7 integrin is an antibody that binds alpha4 beta7 integrin.

12. The method of claim 11, wherein the antibody that binds alpha4 beta7 integrin is vedolizumab.
